(19) United States
(12) Patent Application Publication
(10) Pub. No.: US 2020/0071649 A1
(43) Pub. Date: Mar. 5, 2020

(54) CELL CULTURING SUBSTRATE, PRODUCTION METHOD THEREFOR, AND METHOD FOR CULTURING CELLS

(71) Applicant: Osaka Gas Chemicals Co., Ltd., Osaka-shi, Osaka (JP)

(72) Inventors: Masanobu Okita, Osaka-shi, Osaka (JP); Koh Kidena, Osaka-shi, Osaka (JP); Nan Wu, Osaka-shi, Osaka (JP)

(73) Assignee: Osaka Gas Chemicals Co., Ltd., Osaka-shi, Osaka (JP)

(21) Appl. No.: 16/497,909

(22) PCT Filed: Mar. 29, 2018
(86) PCT No.: PCT/JP2018/013341

§ 371 Date: Sep. 26, 2019

(30) Foreign Application Priority Data
Mar. 30, 2017 (JP) 2017-068483

(51) Int. Cl. C12M 1/12; C12N 5/00

(52) U.S. Cl. CPC C12M 25/14; C12N 5/0068

(57) ABSTRACT

Provided is a cell-culture substrate that can improve cellular activity functions, that is less likely to inactivate cellular activity, and that can be used in a long-term culture. The cell-culture substrate according to the present invention contains hydrophobic silica aerogel. A method for producing the cell-culture substrate according to the present invention includes hydrolyzing a starting material containing a first hydrolyzable silane compound and a second hydrolyzable silane compound that is other than the first hydrolyzable silane compound to produce hydrophobic silica aerogel.

### TECHNICAL FIELD

The present invention relates to a cell-culture substrate usable for cell culture, a method for producing the substrate, and a method for culturing cells.

### BACKGROUND ART

With the growing interest in regenerative medicine, substrates for cell culture have drawn attention, and a variety of cell-culture substrates have been developed.

Cell-culture substrates are known to be able to greatly affect the increase in cell count in a culture, cellular activity characteristics, cell differentiation, etc. From this viewpoint, for example, application of three-dimensionally structured materials in such a cell-culture substrate have also been intensively studied. Specifically, PTL 1 suggests a cell-culture substrate provided with recesses and protrusions on its surface.

### CITATION LIST

### Patent Literature

PTL 1: JP2012-249547

### SUMMARY OF INVENTION

### Technical Problem

However, cell culture using a traditional cell-culture substrate, such as a tissue culture, exhibits decreases or loss in cellular activity functions (e.g., albumin secretion activity of liver cells). Thus, further improvement is needed in functionality of such substrates. In particular, drug development and liver function research using cell culture require the maintenance of cellular activity without inactivating cells. However, it has been difficult to maintain cellular activity for a prolonged. period of time in cell culture using traditional cell-culture substrates.

The present invention was made in view of the circumstances described above, and an object of the invention is to provide a cell-culture substrate that can improve cellular activity functions, that is less likely to inactivate cellular activity, and that is suitable for culturing cells for a prolonged period of time.

### Solution to Problem

The present inventors conducted extensive research to achieve the object, and found that the use of silica aerogel with a specific structure as a substrate can achieve the object, and they completed the present invention.

Specifically, the present invention includes, for example, the subject matter described in the following items.

### Item 1

A cell-culture substrate comprising hydrophobic silica aerogel.

### Item 2

The cell-culture substrate according to Item 1, wherein the hydrophobic silica aerogel has a structure in which at least one hydrocarbon group is bound to a silicon atom.

### Item 3

The cell-culture substrate according to Item 1 or 2, wherein the at least one hydrocarbon group is an alkyl group.

### Item 4

A method for producing a cell-culture substrate, the method comprising

hydrolyzing a starting material containing a first hydrolyzable silane compound and a second hydrolyzable silane compound that is other than the first hydrolyzable silane compound to produce hydrophobic silica aerogel.

### Item 5

The method according to item 4, wherein

the first hydrolyzable silane compound is a trifunctional alkoxysilane,

the second hydrolyzable silane compound is a bifunctional alkoxysilane, and

the bifunctional alkoxysilane is present in an amount of 0.005 mol or more and 0.5 mol or less per mol of the trifunctional alkoxysilane.

### Item 6

A method for culturing cells, the method comprising

culturing cells using the cell-culture substrate of any one of Items 1 to 3.

### Advantageous Effects of Invention

The cell-culture substrate according to the present invention can improve cellular activity functions, and is less likely to inactivate cellular activity and suitable for culturing cells for a prolonged period of time.

### BRIEF DESCRIPTION OF DRAWINGS

FIG. 1 is graphs illustrating a change in cell count (a) and the results of albumin secretion activity (b) in cell culture tests in Test Examples 1-1 and 1-2 (day 3 in culture).

FIG. 2 is graphs illustrating a change in cell count (a) and the results of albumin secretion activity (b) in cell culture tests of Test Examples 2-1 and 2-2 (day 5 and day 10 in culture).

FIG. 3 is scanning microscopic images that illustrate the observation results of cell adhesion in cells that were cultured using hydrophobic aerogel or TCP.

### DESCRIPTION OF EMBODIMENTS

The following describes embodiments of the present invention in detail. In the present specification, the terms “containing” and “including” include the concept of “containing,” “including,” “consisting essentially of,” and “consisting of.”

### 1. Cell-Culture Substrate

The cell-culture substrate according to the present invention contains hydrophobic silica aerogel. The cell-culture substrate according to the present invention is a member for use as a substrate for culturing cells. Due to the presence of hydrophobic silica aerogel, the cell-culture substrate according to the present invention can improve cellular activity functions and is less likely to inactivate cellular activity and. suitable for use in a log-term culture.

Silica aerogel is a material that has the repeating unit “—O—Si—O—” as the basic structure.

Hydrophobic silica aerogel contained in a cell-culture substrate means, for example, that the hydrophobic silica aerogel has higher hydrophobicity than silica aerogel prepared from an alkoxysilane that contains only a tetrafunctional alkoxysilane. Preferably, hydrophobic silica aerogel is insoluble in water.

The carbon content in hydrophobic silica aerogel is, for example, 8 mass % or more, and preferably 8.81 mass % or more. A carbon content of 8 mass % or more can achieve high hydrophobicity.

Hydrophobic silica aerogel preferably has a structure in. which at least one hydrocarbon group is bound to a silicon atom. In this case, hydrophobic silica aerogel can exhibit increased hydrophobicity; thus, the cell-culture substrate can improve cellular activity functions and makes it less likely for cellular activity to be inactivated, enabling the culture for a prolonged period of time.

The type of hydrocarbon group is not particularly limited. The hydrocarbon group may be either a saturated hydrocarbon group or an unsaturated hydrocarbon group. The hydrocarbon group may have a linear structure, a branched structure, or a cyclic structure.

Examples of hydrocarbon groups include alkyl having 1 to 20 carbon atoms, alkenyl having 1 to 20 carbon atoms, and aryl having 6 to 20 carbon atoms. More specific hydrocarbon groups include methyl, ethyl, linear or branched propyl, linear or branched butyl, and phenyl.

The hydrocarbon group is preferably alkyl. In this case, the cell-culture substrate can further improve cellular activity functions and is less likely to inactivate cellular activity, enabling the use of cells in a culture for a prolonged period of time. When the hydrocarbon group is alkyl, the structure of the hydrophobic silica aerogel is likely to be stabilized, and the production of hydrophobic silica aerogel is easy. Particularly

preferably, alkyl has 1 to 3 carbon atoms. In this case, the structure of hydrophobic silica aerogel is easily controlled, and the production of a target hydrophobic silica aerogel is easy.

The shape of hydrophobic silica aerogel is not particularly limited. Examples include a variety of shapes, such as spherical particles, elliptical particles, irregularly shaped particles, fibrous shapes, rod shapes, and. needle shapes. When hydrophobic silica aerogel is in the form of particles, hydrophobic silica aerogel may be, for example, an assembly of many particles. Such an assembly includes a porous assembly, a fibrous assembly, and a networked assembly.

The form of hydrophobic silica aerogel is not particularly limited. Examples include powder, bulks, plates, slime, membranes, and sheets.

The method for producing hydrophobic silica aerogel is not particularly limited. For example, hydrophobic silica aerogel can be produced in accordance with a known method for producing silica aerogel.

For example, hydrophobic silica aerogel can be produced by a method including hydrolyzing a starting material containing a first hydrolyzable silane compound and a second hydrolyzable silane compound. that is other than the first hydrolyzable silane compound. This step is referred to as “step A” below.

A hydrolyzable silane compound refers to a compound. that contains silicon atoms and that is hydrolyzable. Examples of hydrolyzable silane compounds include alkoxysilanes.

The first hydrolyzable silane compound usable in step A includes trifunctional alkoxysilanes.

Examples of trifunctional alkoxysilanes include methyltrimethoxysilane, methyltriethoxysilane, ethyltrimethoxysilane, ethyltriethoxysilane, n-propyltrimethoxysilane, n-propyltrietboxysilane, i-propyltriethoxysilane, phenyltrimethoxysilane, and phenyltriethoxysilane. These alkoxysilanes can be used singly or in a combination of two or more.

The second hydrolyzable silane compound usable in step A includes bifunctional alkoxysilanes. Examples of bifunctional alkoxysilanes include dimethyldimethoxysilane, dimethyldiethoxysilane, diphenyldiethoxysilane, diphenyldimethoxysilane, methylphenyldiethoxysilane, methylphenyldimethoxysilane, diethyldiethoxysilane, and diethyldimethoxysilane. These alkoxysilanes can be used singly or in a combination of two or more.

The starting material for use in step A may contain one or more hydrolyzable silane compounds that are other than the first hydrolyzable silane compound and the second hydrolyzable silane compound. Examples of such hydrolyzable silane compounds include tetrafunctional alkoxysilanes.

Examples of tetrafunctional alkoxysilanes include tetramethoxysilane and tetraethoxysilane. Other alkoxysilanes for use include bis trimethylsilyl methane, bis trimethylsilyl ethane, bis trimethylsilyl hexane, and vinyltrimethoxysilane. These alkoxysilanes can be used singly or in a combination of two or more.

A partial hydrolysate of an alkoxysilane is also usable as the first hydrolyzable silane compound and/or the second hydrolyzable silane compound.

The starting material for use in step A may contain two or more different first hydrolyzable silane compounds. The starting material for use in step A may also contain two or more different second hydrolyzable silane compounds.

The starting material for use in step A is preferably a combination of a trifunctional alkoxysilane for the first hydrolyzable silane compound and a bifunctional alkoxysilane for the second hydrolyzable silane compound. The bifunctional alkoxysilane content in this combination is, for example, 0.001 mol or more, preferably 0.005 mol or more, and more preferably 0.01 mol or more, and, for example, 0.5 mol or less, and preferably 0.15 mol or less, per mol of the trifunctional alkoxysilane. In this case, the resulting cell-culture substrate can further improve cellular activity functions in cell culture, and is less likely to inactivate cellular activity and suitably used in a long-term cell culture. A bifunctional alkoxysilane content per mol of the trifunctional alkoxysilane falling within the range from the upper limit to the lower limit described above makes it likely for the aerogel to become hydrophobic, while making it less likely for transparency to decrease.

The starting material for use in step A may also contain. materials other than the first hydrolyzable silane compound and the second hydrolyzable silane compound.

The conditions for hydrolysis in step A are not particularly limited. For example, conditions equivalent to the conditions for hydrolyzing a known alkoxysilane can be applied. Specifically, a sol-gel process, for example, can be used for hydrolysis.

The sol-gel process includes subjecting a starting material that contains the first hydrolyzable silane compound and the second hydrolyzable silane compound to hydrolysis and condensation polymerization in the presence of a solvent, thereby obtaining a wet, gelled compound with a silica framework.

The solvent for use in the sol-gel process is not particularly limited, and a solvent known to be usable in the sol-gel process can be selected. Examples of such a solvent include water, alcohols, such as methanol and ethanol, acetone, N,N-dimethylformamide, and tetrahydrofuran. The solvent may be a mixture of two or more solvents, or a mixture of water and an organic solvent.

The sol-gel process may be performed in the presence of an acid. Examples of such an acid include hydrochloric acid, citric acid, acetic acid, nitric acid, and sulfuric acid.

The sol-gel process may be performed in the presence of a base. Examples of such a base include ammonia, amine compounds, piperidine, and urea.

The sol-gel process may be performed in the presence of both an acid and a base. The acid and base can be used in the form of, for example, an aqueous solution.

The sol-gel process may also be performed in the presence of other suitable components. Examples of suitable components include known surfactants, dispersion stabilizers, viscosity adjusters, and pH adjusters. A surfactant for use may be anionic, cationic, or nonionic.

The reaction temperature and reaction time for the sol-gel process are not particularly limited. For example, conditions equivalent to known conditions can be applied. The ratio of the starting material to the solvent for use, the amount of optional components, etc. in the sol-gel process are also not particularly limited. Conditions equivalent to those applied in the reaction for preparing known silica aerogel particles can be selected.

The gel obtained in the sol-gel process may optionally be subjected to aging and solvent replacement. The conditions for aging are not particularly limited, and conditions equivalent to known conditions can be applied.

The method for solvent replacement is also not particularly limited. For example, solvent replacement can be performed in accordance with a known method. When performing solvent replacement, the solvent in the gel obtained by the sol-gel process is, for example, preferably replaced with a solvent that can be subjected to supercritical drying described later. Examples of the solvent that can be subjected to supercritical drying described later include alcohols, such as methanol, ethanol, and isopropanol; aromatic compounds, such as benzene, and toluene; and others, such as hydrocarbon solvents, amide solvents, ketone solvents, and ester solvents. Solvent replacement can be performed using different two or more solvents by replacing a solvent with the two or more solvents sequentially. Solvent replacement may be performed multiple times.

After the gel obtained in the sol-gel process is subjected to solvent replacement, the solvent is dried, thereby obtaining hydrophobic silica aerogel. The method for drying the solvent is not particularly limited, and supercritical drying, for example, can be used.

The conditions for supercritical drying are not particularly limited, and a known supercritical drying technique can be used. For example, a known supercritical drying technique using supercritical carbon dioxide can be used.

Before performing supercritical drying, a treatment, such as pulverization treatment, of the gel obtained in the sol-gel process may be performed. Alternatively, after performing supercritical drying, a treatment, such as pulverization treatment, of the gel obtained in the sol-gel process may be performed.

As described above, the production method including step A, optionally with the step of performing solvent replacement and the step of drying the solvent by a supercritical drying technique etc., can provide hydrophobic silica aerogel suitable for a cell-culture substrate.

The cell-culture substrate according to the present invention may contain other materials as long as the effects of the present invention are not impaired. The cell-culture substrate according to the present invention can also be formed only of hydrophobic silica aerogel.

The shape of the cell-culture substrate according to the present invention is not particularly limited, and may be the same as that of a known substrate.

The application of the cell-culture substrate according to the present invention is not particularly limited, and the method for culturing cells using the cell-culture substrate according to the present invention is also not particularly limited. For example, cells can be cultured by seeding a cell-containing medium onto the cell-culture substrate according to the present invention by a known method.

The cell-culture substrate according to the present invention may be subjected to processing treatment (e.g., molding treatment, heating treatment, and surface treatment). For example, the method, such as the surface treatment method, is not particularly limited, and a wide range of surface treatment methods used for known substrates for media can be used. For example, the surface of the cell-culture substrate may be coated with collagen.

The cell-culture substrate according to the present invention may be, for example, kept in or adhered to a container for cell culture.

Silica aerogel is typically highly hydrophilic. Thus, silica aerogel that has not been subjected to any hydrophobizing treatment is not suitable for use in a cell-culture substrate, because such silica aerogel is decreased by, for example, dissolving in water.

In contrast, due to the presence of hydrophobic aerogel, the cell-culture substrate according to the present invention is less likely to be dissolved in water, and is thus suitable for a cell-culture substrate. In particular, performing cell culture using the cell-culture substrate according to the present invention can improve cellular activity functions and is also less likely to inactivate cellular activity. Accordingly, the cell-culture substrate according to the present invention is suitable for use in a long-term cell culture.

In particular, the use of hydrophobic aerogel as a cell-culture substrate can reduce excessive stretching of cells and enables cells to grow three-dimensionally. Thus, the cell-culture substrate according to the present invention is a particularly suitable method for a long-term cell culture. Drug development or liver function research that uses cell culture desires the maintenance of the activity without inactivating cells, and the present invention suitably meets such a need.

The type of the culture fluid for use in cell culture performed using the cell-culture substrate according to the present invention is not particularly limited. For example, the type of the culture fluid can be the same as a known culture fluid. The culture fluid may also contain known components, such as nutritional components for cell culture, such as carbohydrates, lipids, amino acids, minerals, and vitamins; components as growth factors for cell culture; and pH adjusters.

With a medium prepared using the cell-culture substrate according to the present invention, a variety of cells can be cultured. The cells may be stem cells, precursor cells, or functional cells. The “functional cells” as used here refer to differentiated cells, such as liver cells. The cells may be one type of cells or a combination of two or more types of cells.

A kit for cell culture can also be prepared using the cell-culture substrate according to the present invention. Such a kit is suitable for use in culturing cells.

### 2. A Method for Producing a Cell-Culture Substrate

The method for producing the cell-culture substrate according to the present invention is not particularly limited. The cell-culture substrate can be produced using hydrophobic silica aerogel prepared by the same method as the method for producing hydrophobic silica aerogel described above.

The method for producing the cell-culture substrate according to the present invention may include, for example, hydrolyzing a starting material containing a first hydrolyzable silane compound and. a second hydrolyzable silane compound that is other than the first hydrolyzable silane compound to produce hydrophobic silica aerogel. This step is the same as “step A” described above, and the specific aspect is also the same. The solvent replacement and solvent drying method that are optionally added after step A are also as described above.

The cell-culture substrate according to the present invention can be produced using the obtained hydrophobic silica aerogel. For example, hydrophobic silica aerogel as is can be used as a cell-culture substrate. Alternatively, the cell-culture substrate according to the present invention can be produced by optionally subjecting the hydrophobic silica aerogel to molding treatment, coating treatment, etc.

The cell-culture substrate can be produced, for example, in a container for use in cell culture. For example, hydrophobic silica aerogel is produced in a container for use in cell culture in accordance with step A, thereby forming an in-container cell-culture substrate that contains the hydrophobic silica aerogel.

### EXAMPLES

The following describes the present invention with reference to Examples in more detail. However, the present invention is not limited to embodiments of these Examples.

### Example 1

In a Petri dish for cell culture, 0.8 g of hexadecyltrimethylammonium bromide (CTAB), as a surfactant, and 6.0 g of urea were dissolved in 20 g of a 0.05 mol/L acetic acid aqueous solution, and the mixture was stirred for 30 minutes, thereby obtaining mixture solution A.

9.61 g of trimethoxymethylsilane (MTMS, a reagent manufactured by Kanto Chemical Co., Inc.) as the first hydrolyzable silane compound and 0.52 g of dimethoxydimethylsilane as the second hydrolyzable silane compound were dissolved in mixture solution A, and the mixture was stirred for 30 minutes to perform hydrolysis. That is, this operation was performed such that 0.05 mol of the bifunctional alkoxysilane was present per mol of the trifunctional alkoxysilane. Thereafter, the temperature inside the Petri dish was kept at 60° C., and the dish was allowed to stand to turn the content into a gel, followed by allowing the gel to stand for 96 hours, thereby aging the gel. This gel, together with the Petri dish, was then transferred into a 5-fold volume of ethanol and maintained for 6 hours. Ethanol was then removed, and fresh ethanol was added to exchange the solvent. This solvent exchange was repeated 3 times in total, and the gel was transferred into 2-propanol, followed by solvent exchange with 2-propanol every 6 hours in the same manner. This provided a gel whose solvent was replaced with 2-propanol in the Petri dish.

This gel, together with the Petri dish, was kept in a pressure-resistant container and supplied with carbon dioxide, followed by maintaining the pressure-resistant container at 40° C. at 9 MPa for 30 minutes, thereby replacing the 2-propanol in the gel with carbon dioxide. Thereafter, the inside of the pressure-resistant container was adjusted to 80° C. at 14 MPa, which is the supercritical condition for carbon dioxide, and. supercritical drying was performed for about 24 hours to form the target hydrophobic silica aerogel in the Petri dish as the cell-culture substrate.

### Test Example 1-1

The hydrophobic aerogel in the Petri dish prepared in Example 1 was subjected to dry-heat sterilization at 150° C. for 3 hours (a pretreatment), and then to sterilization treatment by irradiating the aerogel with UV for 2 hours. Subsequently, the surface of the hydrophobic aerogel in the Petri dish was coated with 0.3 mg/mL type I collagen. The result was for use as a substrate. A culture test of human hepatoblastoma cells (HepG2) was performed using this substrate.

Culture conditions: 37° C., 5% CO₂ atmosphere (standard culture conditions)
Medium: DMEM+10% FES (culture fluid)
Medium amount: 3 ML
Culture time period: 3 days (Medium replacement was performed on day 1.)
Cell seeding density: 2×10⁵⁵/dish
The diameter of the dish (Petri dish) was 3.5 cm.

### Test Example 1-2

A culture test was performed in the same manner as in Test Example 1-1 except that a tissue culture plate (TCP) was used as substrate instead of the hydrophobic aerogel substrate.

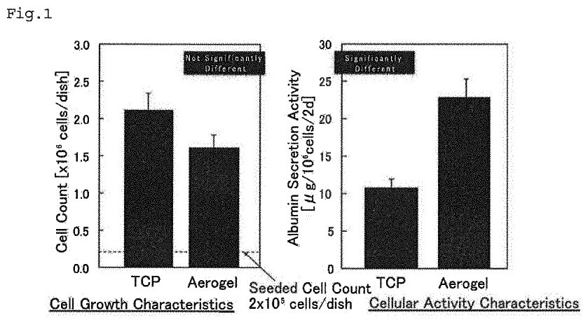

FIG. 1 is graphs illustrating the results of the cell culture tests performed in Test Examples 1-1 and 1-2 (day 3 in each culture). FIG. 1(a) shows a change in cell count, and FIG. 1(b) shows the results of albumin secretion activity.

The albumin secretion activity was measured by quantifying the albumin level secreted into the medium by enzyme-linked immunosorbent assay (ELISA), and converting the result into an. albumin secretion rate per unit cell count. The cell count was measured by a DNA-DAPI (4,6-diaminodino-2-phenylindole, Wako Pure Chemical Industries, Ltd.) fluorescence method. More specifically, a calibration curve was prepared by plotting DNA extracted from predetermined cells and the fluorescence intensity of DNA-DAPI, and calculating the cell count of cultured cells based on the relation. The albumin secretion rate per unit cell count above was calculated based on this cell count.

A comparison between Test Example 1-1 (hydrophobic aerogel) and Test Example 1-2 (TCP) in FIG. 1(a) shows that there is no significant difference in cell growth between Examples 1-1 and 1-2, indicating that the use of hydrophobic aerogel as a substrate has no impact on cell growth. However, FIG. 1(b) reveals that the use of hydrophobic aerogel as a substrate significantly increases albumin secretion activity, compared with the use of TCP.

### Test Example 2-1

A culture test on rat liver cells was performed using the substrate for media prepared in Test Example 1-1.

Culture conditions: 37° C., 5% CO₂ atmosphere (standard culture conditions)
Medium: HDM (culture fluid)
Medium amount: 3 mL
Culture time period: 10 days (Medium replacement was performed every 2 days.)
Cell seeding density: 5×10⁵/dish
The diameter of the dish (Petri dish) was 3.5 cm.

### Test Example 2-2

A culture test was performed in the same manner as in Test Example 2-1 except that a tissue culture plate (TCP) was used as a substrate instead of the hydrophobic aerogel substrate.

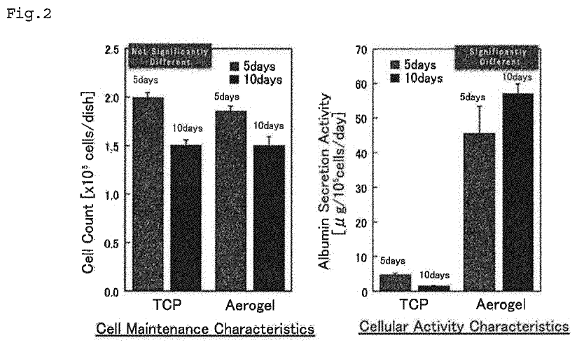

FIG. 2 is graphs illustrating the results of the cell culture tests performed in Examples 2-1 and 2-2 (day 5 and day 10 in culture). FIG. 2(a) shows a change in cell count, and FIG. 2(b) shows the results of albumin secretion activity.

A comparison between Test Example 2-1 (hydrophobic aerogel) and Test Example 2-2 (TCP) in FIG. 2 shows a decrease in cell count in the culture using hydrophobic aerogel as a substrate, compared with the seeded cell count before starting culture; however, there was no difference in maintenance (i.e., no decreasing trend with time). This indicates that the use of hydrophobic aerogel as a substrate has no impact on cell growth.

When the cells were cultured for 5 days and 10 days, there was a noticeable difference in albumin secretion activity between hydrophobic aerogel and TOP. In particular, the substrate containing hydrophobic aerogel was confirmed to be suitable for use in a long-term cell culture.

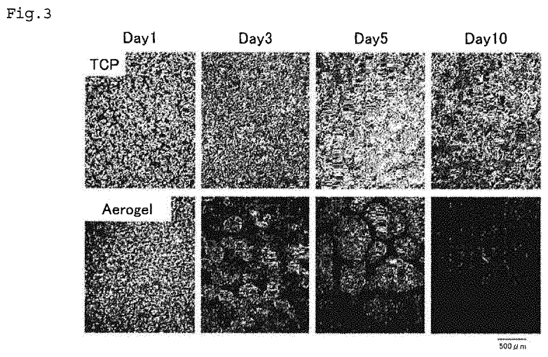

FIG. 3 is microscopic phase-contrast images showing the observation results of cell adhesion in culturing using hydrophobic aerogel or TCP on days 1, 3, 5, and 10 in culture. (The four photographs in the upper row are of the culture using TCP, and the four photographs in the lower row are of the culture using hydrophobic aerogel.)

The images of FIG. 3 suggest that the use of hydrophobic aerogel as a substrate leads to weak cell stretching, inducing aggregation. More specifically, the use of hydrophobic aerogel as a substrate reduces excessive stretching of cells, thus allowing the cells to grow three-dimensionally. This indicates that a substrate containing hydrophobic aerogel is suitable for use in a long-term cell culture.

The results above demonstrate that the cell-culture substrate containing hydrophobic aerogel according to the present invention can improve cellular activity functions in cell culture, and is less likely to inactivate cellular activity. The cell-culture substrate containing hydrophobic aerogel is thus suitable for use in a long-term cell culture.

We claim:

1. A cell-culture substrate comprising hydrophobic silica aerogel.

2. The cell-culture substrate according to claim 1, wherein the hydrophobic silica aerogel has a structure in which at least one hydrocarbon group is bound to a silicon atom.

3. The cell-culture substrate according to claim 1, wherein the at least one hydrocarbon group is an alkyl group.

4. A method for producing a cell-culture substrate, the method comprising
hydrolyzing a starting material containing a first hydrolyzable silane compound and a second hydrolyzable silane compound that is other than the first hydrolyzable silane compound to produce hydrophobic silica aerogel.

5. The method according to claim 4, wherein the
first hydrolyzable silane compound is a trifunctional alkoxysilane,
the second hydrolyzable silane compound is a bifunctional alkoxysilane, and the bifunctional alkoxysilane is present in an amount of 0.005 mol or more and 0.5 mol or less per mol of the trifunctional alkoxysilane.

6. A method for culturing cells, the method comprising
culturing cells using the cell-culture substrate of claim I.

7-9. (canceled)
